(19) United States
(12) Patent Application Publication
(10) Pub. No.: US 2024/0409667 A1
(43) Pub. Date: Dec. 12, 2024

(54) INHIBITION OF CONTACT PATHWAY ACTIVATION FOR THE TREATMENT OF NEUROINFLAMMATION

(71) Applicants: Oregon Health & Science University, Portland, OR (US); The United States Government as represented by the Department of Veterans Affairs, Washington, DC (US)

(72) Inventors: Owen McCarty, Portland, OR (US); Erik Tucker, Portland, OR (US); Norah Verbout, Portland, OR (US); Halina Offner, Portland, OR (US); Tia Kohs, Portland, OR (US)

(73) Assignees: Oregon Health & Science University, Portland, OR (US); The United States Government as represented by the Department of Veterans Affairs, Washington, DC (US)

(21) Appl. No.: 18/733,620

(22) Filed: Jun. 4, 2024

Related U.S. Application Data
(60) Provisional application No. 63/506,641, filed on Jun. 7, 2023.

(51) Int. Cl. C07K 16/40; A61K 39/00; A61P 25/28

(52) U.S. Cl. CPC C07K 16/40; A61P 25/28; A61K 2039/505; C07K 2317/565

(57) ABSTRACT

Methods of treating neuroinflammation in a subject by administering to the subject an agent that inhibits activation of coagulation factor XI (FXI), inhibits the activity of activated FXI (FXIa), or reduces expression of FXI are described. The agent that inhibits activation of FXI can be a monoclonal antibody specific for FXI, such as a monoclonal antibody having the complementarity determining region (CDR) sequences of anti-FXI antibody 14E11. Small molecule inhibitors and antisense compounds directed to FXI or FXIa are also contemplated as agents that inhibit activity of FXIa or reduce expression of FXI. The neuroinflammation in the subject can be associated with any one of a number of different diseases or disorders, such as an autoimmune disease of the central nervous system (CNS) or a neurodegenerative disease.

### CROSS REFERENCE TO RELATED APPLICATIONS

This application claims the benefit of U.S. Provisional Application No. 63/506,641, filed Jun. 7, 2023, which is herein incorporated by reference in its entirety.

### ACKNOWLEDGMENT OF GOVERNMENT SUPPORT

This invention was made with government support under R01 HL101972 awarded by the National Institutes of Health. The government has certain rights in the invention.

### FIELD

This disclosure concerns the treatment of neuroinflammation, such as neuroinflammation in a subject with an autoimmune or neurodegenerative disease, using an agent that inhibits activation of coagulation factor XI (FXI), inhibits activity of activated FXI, or inhibits expression of FXI.

### INCORPORATION OF ELECTRONIC SEQUENCE LISTING

The electronic sequence listing, submitted herewith as an XML file named 899-110226-02.xml (10,443 bytes), created on May 22, 2024, is herein incorporated by reference in its entirety.

### BACKGROUND

Multiple sclerosis (MS) is a neurodegenerative, demyelinating disease of the central nervous system (CNS) that affects approximately 2.2 million individuals globally. MS is characterized by inflammatory lesions, axonal damage, and leukocyte trafficking across the blood-brain barrier (BBB). The pathophysiology of MS is believed to be caused in part by increased BBB vascular permeability, leading to the transmigration of peripheral T cells into the brain parenchyma that induce lesions and subsequent demyelination.

In addition to immune cells, other factors contribute to the pathophysiology of MS. Clinical observations of MS patients and studies using animal models suggest a connection between the coagulation cascade and inflammatory processes in MS. The coagulation cascade is a series of enzymatic reactions in which a zymogen precursor becomes activated to its enzyme form, which then catalyzes the next reaction in the pathway, ultimately activating thrombin and forming a fibrin clot.

A need exists for identifying therapeutic interventions that prevent or diminish the pathophysiology associated with MS and other neurodegenerative diseases.

### SUMMARY

Coagulation factor XI (FXI) is a major substrate of activated FXII (FXIIa), the initiator of the intrinsic pathway of coagulation. Decreasing or eliminating FXI activity through gene knockout, pharmacologic inhibition, or antisense oligonucleotide mediated knockdown is antithrombotic without impairing hemostasis. The present disclosure demonstrates that pharmacological inhibition of FXI reduces disease severity, immune cell migration, axonal damage, and BBB disruption in an animal model of MS. These data support the use of inhibitors of FXI as therapeutic agents for the treatment of neuroinflammation.

Provided herein are methods of treating neuroinflammation in a subject by administering to the subject a therapeutically effective amount of an agent that inhibits activation of coagulation factor XI (FXI), inhibits the activity of activated FXI (FXIa), or reduces expression of FXI. The agent can be, for example, a monoclonal antibody, a small molecule inhibitor, or an antisense compound.

In some aspects of the disclosed methods, the agent that inhibits activation of FXI is a monoclonal antibody specific for FXI, such as a monoclonal antibody having the complementarity determining region (CDR) sequences of anti-FXI antibody 14E11. In other aspects, the agent that inhibits activity of activated FXI is a small molecule that inhibits the activated form of FXI (FXIa), such as asundexian or milvexian. In yet other aspects, the agent that reduces expression of FXI is an antisense compound targeting a FXI nucleic acid molecule, such as an FXI mRNA.

In some aspects, the agent is administered in a composition that includes a pharmaceutically acceptable carrier. In some examples, the composition is formulated for intravenous administration.

In some aspects of the disclosed methods, the subject has an autoimmune disease of the central nervous system (CNS), such as but not limited to multiple sclerosis (MS), neuromyelitis optica (NMO), anti-myelin oligodendrocyte glycoprotein antibody disease (MOG), autoimmune encephalitis, acute disseminated encephalomyelitis (ADEM), chronic meningitis, central nervous system vasculitis, or Hashimoto's encephalitis. In particular examples, the autoimmune disease is MS.

In some aspects of the disclosed methods, the subject has a neurodegenerative disease, such as but not limited to amyotrophic lateral sclerosis (ALS), Parkinson's disease, Alzheimer's disease, Huntington's disease, Lewy body dementia, vascular dementia, or ischemic stroke.

The foregoing and other features of this disclosure will become more apparent from the following detailed description of several aspects which proceeds with reference to the accompanying figures.

### BRIEF DESCRIPTION OF THE DRAWINGS

FIGS. 1A-1C: Pharmacologic targeting of FXI with 14E11 attenuates experimental autoimmune encephalomyelitis (EAE) symptoms. Male C57BL/6 mice immunized with MOG/CFA/Ptx were monitored and assigned a disease score daily. At peak disease onset (disease score ≥2.5), mAb 14E11 (1 mg/kg, i.v.) was administered every other day for four days (FIG. 1A). The mean disease scores (FIG. 1B) and the cumulative disease index (FIG. 1C) for 14E11-treated mice were significantly lower compared to mice treated with vehicle. Data are presented as mean±SEM for two independent experiments, where n=7-9. Statistical analyses were performed using Mann-Whitney tests on GraphPad Prism 9. Statistical significance is indicated by a single asterisk (*) for P≤0.05.

FIGS. 2A-2B: EAE-induced inflammation in the spleen is not affected by treatment with 14E11. Splenocytes were obtained from mice with EAE treated with either vehicle or 14E11 (1 mg/kg, intravenous). The tissues were homogenized and single cell suspensions were analyzed by flow cytometry. Cells were gated for CD11b⁺ and assayed for TNFα (FIG. 2A) and ICAM-1 (FIG. 2B) to study the macrophage subpopulation phenotype. Data are presented as mean±SEM from n=3. Statistical analyses were conducted using unpaired t-tests on GraphPad Prism 9. Statistical significance is indicated by a single asterisk (*) for P≤0.05.

FIGS. 3A-3B: Axonal damage and demyelination in the thoracic spinal cord is reduced following treatment with 14E11. Damage to the myelin in spinal cord sections was assessed by staining the tissues with toluidine blue and using light microscopy to image the semithin sections at 20×. A complete view of the spinal cord cross section was obtained by manually stitching individual images into composites using Adobe Photoshop. The representative images shown are from mice treated with either vehicle (FIG. 3A) or 14E11 (FIG. 3B). Tissue damage in the white matter is denoted. The scale bar in the composite image indicates 100 μm, and the scale bar in the magnified view (63×) indicates 10 μm.

FIGS. 4A-4B: Effects of pharmacologic targeting of FXI by 14E11 treatment on fibrin(ogen) deposition in the spinal cord. Sections of the lumbar region of the spinal cord were stained for fibrin(ogen) deposition from one representative animal per treatment group. 20× images were manually stitched into composites and a threshold for background fluorescence was applied for vehicle (FIG. 4A) or 14E11 treatment (FIG. 4B). Representative images show a reduction in area positive for fibrin(ogen) signal as well as corrected fluorescent integrated density for 14E11 treatment compared to vehicle control. Scale bar indicates 100 μm.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.
SEQ ID NO: 1 is the amino acid sequence of the 14E11 VH domain.
SEQ ID NO: 2 is the amino acid sequence of the 14E11 H-CDR1.
SEQ ID NO: 3 is the amino acid sequence of the 14E11 H-CDR2.
SEQ ID NO: 4 is the amino acid sequence of the 14E11 H-CDR3.
SEQ ID NO: 5 is the amino acid sequence of the 14E11 VL domain.
SEQ ID NO: 6 is the amino acid sequence of the 14E11 L-CDR1.
SEQ ID NO: 7 is the amino acid sequence of the 14E11 L-CDR2.
SEQ ID NO: 8 is the amino acid sequence of the 14E11 L-CDR3.
SEQ ID NO: 9 is a nucleotide sequence encoding the 14E11 VH domain.
SEQ ID NO: 10 is a nucleotide sequence encoding the 14E11 VL domain.

### DETAILED DESCRIPTION

### I. Introduction

Multiple sclerosis (MS) is the most common cause of non-traumatic disability in young adults worldwide. MS pathophysiology includes the formation of inflammatory lesions, axonal damage and demyelination, and blood brain barrier (BBB) disruption. Coagulation proteins, including FXII, can serve as important mediators of the adaptive immune response during neuroinflammation. Indeed, plasma FXII levels are increased during relapse in relapsing-remitting MS patients, and previous studies showed that reducing FXII levels was protective in an experimental autoimmune encephalomyelitis (EAE) murine model of MS.

The study disclosed herein was designed to determine if pharmacological targeting of FXI, a major substrate of activated FXII (FXIIa), improves neurological function and attenuates CNS damage in an animal model of MS. EAE was induced in male mice using murine myelin oligodendrocyte glycoprotein peptides combined with heat-inactivated Mycobacterium tuberculosis and pertussis toxin. Upon onset of symptoms, mice were treated every other day intravenously with an anti-FXI antibody (14E11) or saline. Antibody 14E11 prevents activation of FXI by FXII (see WO 2010/080623). Disease scores were recorded daily until euthanasia for ex vivo analyses of inflammation. Following treatment with 14E11, there was a reduction in the clinical severity of EAE symptoms, axonal damage and demyelination, and fibrin(ogen) accumulation in the CNS. This indicates that FXI is a suitable therapeutic target for treating the progression of autoimmune disorders of the CNS and other autoimmune and/or neurodegenerative diseases.

In particular, it is shown herein that pharmacological targeting of FXI improved disease scores of EAE. Treatment with 14E11 significantly attenuated clinical symptom severity over the course of eight days, which corresponded to an approximately 40% reduction in the cumulative disease index.

Treatment with 14E11 also resulted in a reduction in EAE-induced inflammation. During the early stages of MS, fibrinogen infiltrates the CNS and activates microglia through the integrin receptor, CD11b/CD18 (Davalos and Akassoglou, Semin Immunopathol 34 (1): 43-62, 2012), resulting in an MI-like activation and pro-inflammatory phenotype (Adams et al., J Exp Med 204 (3): 571-582, 2007; Davalos and Akassoglou, Semin Immunopathol 34 (1): 43-62, 2012; Ryu et al., Nat Comm 6 (1): 8164-8164, 2015). Therefore, CD11b⁺ macrophage expression of TNFα and ICAM-1 were measured as markers of activated macrophages/microglia found to be causative in early stages of EAE. The population of activated macrophages/microglia (CD11b/CD45^{high} cells) and CD4+ T cells in pooled murine brain samples was quantified. Not only did treatment with 14E11 reduce the total number of mononuclear cells in the brain by 50%, but it also reduced percentages of activated CNS macrophages/microglia and T cells. These results strongly support the conclusion that inhibition of FXI plays an important role in limiting EAE-induced inflammation.

Additionally, 14E11-treated mice had reduced demyelination in the white matter of spinal cord tissue compared to the vehicle cohort. FXI inhibition also reduced BBB disruption, as measured by fibrin(ogen) accumulation in the spinal cord.

Overall, pharmacological inhibition of FXI decreased severity of EAE clinical signs, axonal damage, and fibrin(ogen) deposition within the CNS. These data demonstrate that pharmacological inhibition of FXI, such as by treatment with antibody 14E11, reduces disease severity, immune cell migration, axonal damage, and BBB disruption in an animal model of MS, thereby providing an effective therapeutic for treating autoimmune and neurodegenerative disorders.

### II. Abbreviations

ALS amyotrophic lateral sclerosis
BBB blood brain barrier
CNS central nervous system
CDI cumulative disease index
EAE experimental autoimmune encephalomyelitis
F factor
FX coagulation factor X
FXa activated coagulation factor X
FXI coagulation factor XI
FXII coagulation factor XII
FXIIa activated coagulation factor FXII
MMP matrix metalloproteinases
MOG35-55 mouse myelin oligodendrocyte glycoprotein, peptides 35-55
MS multiple sclerosis
OPC oligodendrocyte progenitor cell
PPMS primary progressive multiple sclerosis
Ptx pertussis toxin
RRMS relapsing-remitting multiple sclerosis
SPMS secondary-progressive multiple sclerosis

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: To provide or give a subject an agent, such as an antibody, small molecule, or antisense compound, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen, such as FXI or activated FXI. As used herein, the term “antibody” includes but is not limited to immunoglobulin molecules and antigen-binding fragments, such as single-domain antibodies, Fab fragments, Fab′ fragments, F(ab)′₂ fragments, single chain variable fragments (scFv) and disulfide stabilized variable fragments (dsFv). Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles and is functionally similar to mammalian IgG and IgE. IgW and IgNAR antibodies are produced by cartilaginous fish, while IgX antibodies are found in amphibians.

Antibody variable regions contain framework regions (FR) and hypervariable (HV) regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and A1-Lazikani et al., JMB 273,927-948, 1997; the “Chothia” numbering scheme), Kunik et al. (see Kunik et al., PLOS Comput Biol 8:01002388, 2012; and Kunik et al., Nucleic Acids Res 40 (Web Server issue): W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Shark V_{NAR} are comprised of the following regions (N-terminal to C-terminal): FR1-CDR1-FR2-HV2-FR3a-HV4-FR3b-CDR3-FR4. The positions of CDR1 and CDR3 of V_{NAR} antibodies can be determined, for example, using IMGT. HV2 and HV4 can be determined, for example, using annotation described in Stanfield et al. (Science 305:1770-1773, 2004) and Fennell et al. (J Mol Biol 400:155-170, 2010). Camelid V_{H}H antibodies are produced by several species including camel, llama, alpaca, dromedary, and guanaco, which produce heavy chain antibodies that are naturally devoid of light chains. Camel V_{H}H are comprised of the following regions (N-terminal to C-terminal): FR1-CDR1-FR2-CDR2-FR3-CDR3-FR4. Camel V_{H}H CDR residues can be determined, for example, according to IMGT, Kabat or Paratome.

A “monoclonal antibody” is an antibody produced by a single clone of lymphocytes or by a cell into which the coding sequence of a single antibody has been transfected. Monoclonal antibodies include, for example, humanized monoclonal antibodies, chimeric antibodies and fully human antibodies.

A “chimeric antibody” has framework residues from one species, such as human, and CDRs (which generally confer antigen binding) from another species. A “humanized” antibody is an immunoglobulin including a human framework region and one or more CDRs from a non-human (for example a camel, llama, mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antisense compound: An oligomeric compound that is at least partially complementary to the region of a target nucleic acid molecule to which it hybridizes (such as a FXI nucleic acid molecule). As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. Nonlimiting examples of antisense compounds include primers, probes, antisense oligonucleotides, small interfering RNAs (siRNAs), microRNAs (miRNAs), short hairpin RNAs (shRNAs) and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound.

Antisense oligonucleotide (ASO): A synthetic, single-stranded nucleic acid-based oligomer that is complementary to a target nucleic acid molecule, such as a target RNA. An antisense oligonucleotide can include one or more chemical modifications to the sugar, base, and/or internucleoside linkages. Exemplary modifications to the sugar include, but are not limited to, LNA, 2′-O-methyl, 2′-O-methoxy-ethyl (MOE), 2′-fluoro, cEt and tc-DNA. Modifications to the internucleoside linkages include, for example, phosphorothioate and phosphoramidate. One example of a modified base is 5-methylcytosine.

Autoimmune disease: A disorder in which the immune system produces an immune response (e.g., a B cell or a T cell response) against an endogenous antigen, with consequent injury to tissues. An autoimmune disease of the CNS refers to an autoimmune disease that primarily affects the CNS (e.g., an autoimmune disease that results in autoantibodies directed against cell-surface or intracellular neuronal proteins). Non-limiting examples of autoimmune diseases of the CNS include multiple sclerosis (MS), neuromyelitis optica (NMO), anti-myelin oligodendrocyte glycoprotein antibody disease (MOG), autoimmune encephalitis, acute disseminated encephalomyelitis (ADEM), chronic meningitis, central nervous system vasculitis, and Hashimoto's encephalitis.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. Camel (V_{H}H) single-domain antibodies, V_{H} single-domain antibodies and V_{L} single-domain antibodies contain three CDRs, referred to as CDR1, CDR2 and CDR3.

Conservative variants: As used herein, conservative amino acid substitutions are those substitutions that do not substantially affect or decrease the activity or function of a protein, such as the affinity of an antibody to FXI. For example, an antibody that specifically binds FXI can include at most about 1, at most about 2, at most about 5, and most about 10, or at most about 15 conservative substitutions and specifically bind a FXI polypeptide. The term conservative variation also includes the use of a substituted amino acid in place of an unsubstituted parent amino acid, provided that antibody specifically binds FXI. Non-conservative substitutions are those that reduce an activity or binding to FXI.

Conservative amino acid substitution tables providing functionally similar amino acids are well-known. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Demyelination: Loss of or damage to myelin, or the impairment in growth or development of the myelin sheath. Demyelination inhibits the conduction of signals in the affected nerves, causing impairment in sensation, movement, cognition, or other functions for which nerves are involved. Demyelinating diseases have a number of different causes and can be hereditary or acquired. In some cases, a demyelinating disease is caused by an infectious agent, an autoimmune response, a toxic agent or traumatic injury. In other cases, the cause of the demyelinating disease is unknown or develops from a combination of factors.

Encephalopathy: A disease or disorder of the brain, such as a disease or disorder that alters brain function or structure.

Experimental autoimmune encephalomyelitis (EAE): An animal model of MS (for example, see Gold et al., Brain 129:1953-1971, 2006). EAE animals exhibit characteristic plaques of tissue injury disseminated throughout the CNS. Plaques show infiltration of nervous tissue by lymphocytes, plasma cells, and macrophages, which cause destruction of the myelin sheaths that surround nerve cell axons in the brain and spinal cord. In some cases, EAE is induced by immunization of susceptible animals, such as mice, rats, guinea pigs, or non-human primates, with either myelin or various components of myelin. For example, EAE can be induced by immunization with components of the myelin sheath, such as myelin basic protein, proteolipid protein, or myelin oligodendrocyte glycoprotein (MOG). EAE is a useful and widely accepted model for studying mechanisms of autoimmune CNS tissue injury and for testing potential therapies for MS. EAE also includes “passive EAE” which is induced in the same manner in donor animals but involves the transfer of activated T cells harvested from the donor animal's lymph nodes to naïve recipient animals.

Factor XI (FXI): A zymogen form of activated factor XI (FXIa), an enzyme involved in coagulation. The FXII protein includes four apple domains (A1, A2, A3 and A4) and a catalytic domain (see Gailani and Smith, J Thromb Haemost 7 (Suppl 1): 75-78, 2009). This protein is found only in mammals. FXI is activated by FXIIa, thrombin and via an autocatalytic mechanism. FXI is synthesized as a single polypeptide chain but circulates as a homodimer formed by disulfide bonds. Each polypeptide chain of FXI is approximately 80 kD. During activation of factor XI, an internal peptide bond is cleaved by factor XIIa in each of the two chains, resulting in activated FXI (FXIa), a serine protease composed of two heavy and two light chains held together by disulfide bonds. Activated FXI triggers the middle phase of the intrinsic pathway of blood coagulation by activating factor IX. Defects in this factor lead to Rosenthal syndrome (also known as hemophilia C), a blood coagulation abnormality. The FXI protein is encoded by the F11 gene. FXI is also known as coagulation factor XI or plasma thromboplastin antecedent. As used herein, “coagulation factor XI,” “factor XI” or “FXI” refers to any FXI from any mammalian species that expresses the protein. For example, FXI can be human, non-human primate (such as baboon), mouse, dog, cat, cow, horse, pig, rabbit, raccoon, tiger, anteater, elephant (such as African elephant or Asian elephant), or llama. As used herein, an agent that inhibits activation of FXI refers to any compound that prevents or inhibits the conversion of zymogen FXI to activated FXI (FXIa). An agent that inhibits the activity of activated FXI refers to any agent that inhibits the enzymatic activity of the active form of FXI (FXIa). An agent that reduces expression of FXI refers to any compound that reduces the amount of FXI protein in a cell or subject. Agents that reduce expression of FXI include, for example, antisense compounds that target FXI nucleic acid for destruction, or inhibit translation of FXI.

Framework region: Amino acid sequences interposed between CDRs (and/or hypervariable regions).

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, NY, 1989, chapters 9 and 11.

Inhibits expression or activity: An agent that inhibits expression or activity of FXI (or FXIa) is a compound that reduces the level of FXI mRNA or protein in a cell or tissue, or reduces (including eliminates) one or more activities of FXI/FXIa. For example, an antisense compound targeting FXI inhibits expression of FX1 by promoting the degradation of FXI mRNA, thereby reducing the level of FXI protein. In some aspects, FXI expression is inhibited at least 10%, at least 25%, at least 50%, at least 75%, at least 90%, or at least 95% relative to a control, such as untreated control cells. As another example, an antibody or small molecule that specifically binds or targets FXI or FXIa may inhibit activity of FXI or FXIa by preventing FXI or FXIa protein from interacting with another protein. In some aspects, FXI or FXIa activity is inhibited at least 10%, at least 25%, at least 50%, at least 75%, at least 90%, or at least 95% relative to an untreated control.

Inflammation: A localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, cither systemically or locally at the site of inflammation. The inflammatory response may be measured by many well-known methods, such as the number of white blood cells, the number of polymorphonuclear neutrophils (PMN), a measure of the degree of PMN activation, such as luminal enhanced chemiluminescence, or a measure of the amount of cytokines present. A primary inflammation disorder is a disorder that is caused by the inflammation itself. A secondary inflammation disorder is inflammation that is the result of another disorder. Inflammation can lead to a host of inflammatory diseases, including, but not limited to rheumatoid arthritis, osteoarthritis, inflammatory lung disease (including chronic obstructive pulmonary lung disease), inflammatory bowel disease (including ulcerative colitis and Crohn's Disease), periodontal disease, polymyalgia rheumatica, atherosclerosis, systemic lupus erythematosus, systemic sclerosis, Sjögren's Syndrome, asthma, allergic rhinitis, and skin disorders (including dermatomyositis and psoriasis) and the like. Auto-immune disorders which include an inflammatory component (including, but not limited to multiple sclerosis) are also considered to be inflammatory disorders.

MicroRNA (miRNA): Single-stranded RNA molecules that regulate gene expression. miRNAs are generally 21-23 nucleotides in length. miRNAs are processed from primary transcripts known as pri-miRNA to short stem-loop structures called pre-miRNA and finally to functional miRNA. Mature miRNA molecules are partially complementary to one or more messenger RNA molecules, and their primary function is to down-regulate gene expression. MicroRNAs regulate gene expression through the RNAi pathway.

Multiple sclerosis: An autoimmune disease classically described as a central nervous system white matter disorder disseminated in time and space that presents as relapsing-remitting illness in 80-85% of patients. Diagnosis can be made by brain and spinal cord magnetic resonance imaging (MRI), analysis of somatosensory evoked potentials, and analysis of cerebrospinal fluid to detect increased amounts of immunoglobulin or oligoclonal bands. MRI is a particularly sensitive diagnostic tool. MRI abnormalities indicating the presence or progression of MS include hyperintense white matter signals on T2-weighted and fluid attenuated inversion recovery images, gadolinium enhancement of active lesions, hypointensive “black holes” (representing gliosis and axonal pathology), and brain atrophy on T1-weighted studies. Serial MRI studies can be used to indicate disease progression. Relapsing-remitting multiple sclerosis (RRMS) is a clinical course of MS that is characterized by clearly defined, acute attacks with full or partial recovery and no disease progression between attacks. Secondary-progressive multiple sclerosis (SPMS) is a clinical course of MS that initially is relapsing-remitting, and then becomes progressive at a variable rate, possibly with an occasional relapse and minor remission. Primary progressive multiple sclerosis (PPMS) presents initially in the progressive form.

Neurodegenerative disease: Any type of disease or disorder that is associated with a progressive loss of motor, sensory and/or perceptual functions, and often involves behavioral and cognitive deficits. Neurodegenerative diseases are typically characterized by the progressive loss of structure or function of neurons, such as neurons within the cerebral cortex, basal ganglia, cerebellum, brain stem or motor systems. Neurodegenerative disorders include, but are not limited to, Alzheimer's disease, Parkinson's disease, Huntington's disease, ALS, multiple sclerosis, Lewy body dementia, vascular dementia, progressive supranuclear palsy, corticobasal degeneration, multiple system atrophy and frontotemporal dementia.

Neuroinflammation: Inflammation of the nervous tissue. Neuroinflammation can occur in response to a variety of different cues, including infection, traumatic brain injury, toxic metabolites, aging or autoimmunity (for example, multiple sclerosis). Neuroinflammation is typically chronic and results from sustained activation of glial cells and recruitment of other immune cells into the brain. The central nervous system is typically an immunologically privileged site because peripheral immune cells are generally blocked by the blood brain barrier (BBB). However, circulating peripheral immune cells may surpass a compromised BBB and encounter neurons and glial cells expressing major histocompatibility complex molecules, perpetuating the immune response.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of a therapeutic agent (such as an antibody, small molecule or antisense compound). In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease, such as an autoimmune disease of the CNS or a neurodegenerative disorder.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds FXI are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95% depending on their similarity to the reference sequence. Methods for determining sequence identity over such short windows are available at the NCBI website on the internet. A skilled person will appreciate that these sequence identity ranges are provided for guidance only; it is entirely possible that strongly significant homologs could be obtained that fall outside of the ranges provided.

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule: A molecule, typically with a molecular weight less than about 1000 Daltons, or in some aspects, less than about 750 Daltons or less than about 500 Daltons, wherein the molecule is capable of modulating, to some measurable extent, an activity of a target molecule (such as FXI or FXIa). In some aspects of the disclosed methods, the small molecule is asundexian (BAY2433334) or milvexian (BMS-986177/JNJ-70033093), which are known inhibitors of activated FXI (see, e.g., Piccini et al., Lancet 399 (10333): 1383-1390, 2022; Kubitza et al., Br J Clin Pharmacol 88 (7): 3447-3462, 2022; Weitz et al., N Engl J Med 385:2161-2172, 2021; Wong et al., J Thromb Haemost 20 (2): 399-408, 2022).

Subject: A living multicellular vertebrate organism, a category that includes, for example, mammals and birds. A “mammal” includes both human and non-human mammals, such as mice or non-human primates. In some examples, a subject is a patient, such as a patient that has or is at risk of developing multiple sclerosis or another autoimmune disorder and/or a neurodegenerative disorder.

Therapeutically effective amount: The amount of agent, such as an antibody, small molecule or antisense compound that is alone (or in combination with other therapeutic agents) sufficient to prevent, treat, reduce and/or ameliorate the symptoms and/or underlying causes of a disease or disorder, for example to prevent, inhibit, and/or treat neuroinflammation, such as neuroinflammation resulting from an autoimmune disorder of the CNS (e.g., MS) or a neurodegenerative disorder.

In some aspects, a therapeutically effective amount is sufficient to reduce or eliminate a symptom of a disease, such as MS. For instance, this can be the amount necessary to inhibit or suppress neuroinflammation, demyelination, BBB disruption, axonal damage, fibrin(ogen) deposition, or overall disease severity. In one aspect, a therapeutically effective amount is the amount necessary to eliminate, reduce, or prevent neuroinflammation, such as by at least 10%, at least 20%, at least 30%, at least 40%, at least 50%, at least 60%, at least 70%, at least 75%, at least 80%, at least 90%, at least 95%, or even 100%, such as compared to neuroinflammation prior to treatment or following treatment with another therapeutic agent. In another aspect, a therapeutically effective amount is the amount necessary to reduce the cumulative disease index, such as by at least 10%, at least 20%, at least 30%, at least 40%, at least 50%, at least 60%, at least 70%, at least 75%, at least 80%, at least 90%, at least 95%, or even 100%, for example as compared to the cumulative disease index prior to treatment (or for example as compared to another treatment). In other aspects, a therapeutically effective amount is the amount necessary to reduce, prevent or eliminate axonal damage, demyelination, fibrin(ogen) deposition, and/or BBB disruption, such as by at least 10%, at least 20%, at least 30%, at least 40%, at least 50%, at least 60%, at least 70%, at least 75%, at least 80%, at least 90%, at least 95%, or even 100%, such as compared to axonal damage, demyelination, fibrin(ogen) deposition, and/or BBB disruption prior to treatment or following treatment with another therapeutic agent. When administered to a subject, a dosage will generally be used that will achieve target tissue concentrations (for example, in the CNS) that has been shown to achieve a desired in vitro effect.

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

### IV. Methods of Treating Neuroinflammation

The present disclosure evaluates pharmacological inhibition of FXI in an accepted animal model of multiple sclerosis (MS). Experimental autoimmune encephalomyelitis (EAE) mice were administered a monoclonal antibody (14E11) that specifically binds FXI and prevents its activation by FXII (WO 2010/080623). The results disclosed herein demonstrate that pharmacological inhibition of FXI in EAE animals significantly reduced the overall clinical severity of disease, and specifically reduced EAE-induced inflammation, immune cell migration, demyelination, fibrin(ogen) accumulation, and BBB disruption. These data support the use of agents that inhibit FXI activation, inhibit activity of activated FXI, and/or inhibit expression of FXI as therapeutic agents for the treatment of neuroinflammation, such as neuroinflammation in subjects with autoimmune and/or neurological diseases.

Provided herein are methods of treating neuroinflammation in a subject by administering to the subject a therapeutically effective amount of an agent that inhibits activation of FXI, inhibits the activity of activated FXI (FXIa), or reduces expression of FXI. In some aspects, the method further includes selecting a subject for treatment, such as a subject with an autoimmune disorder of the CNS or a neurodegenerative disorder.

In some aspects, the agent that inhibits activation of FXI, inhibits activity of FXIa, or reduces expression of FXI is a monoclonal antibody, a small molecule inhibitor, or an antisense compound.

In particular aspects, the agent that inhibits activation of FXI is a monoclonal antibody specific for FXI, such as a monoclonal antibody that blocks activation of FXI by activated FXII (FXIIa). The monoclonal antibody specific for FXI can bind, for example, the A2 domain of FXI. In several examples, the FXI-specific monoclonal antibody does not block activation of FXI by thrombin or tissue factor and/or does not block activation of prekallikrein by FXIIa. In some examples, the monoclonal antibody specific for FXI has one or more complementarity determining region (CDR) sequences (such as 1, 2, 3, 4, 5 or all 6 CDR sequences) from antibody 14E11, which has a VH domain sequence that includes the amino acid sequence of SEQ ID NO: 1 and a VL domain sequence that includes the amino acid sequence of SEQ ID NO: 5. In some examples, the VH domain includes the heavy chain complementarity determining region 1 (HCDR1), HCDR2 and HCDR3 sequences of SEQ ID NO: 1; and the VL domain includes the light chain complementarity determining region 1 (LCDR1), LCDR2, and LCDR3 sequence of SEQ ID NO: 5. In particular examples, the amino acid sequences of the HCDR1, HCDR2 and HCDR3 respectively include SEQ ID NO: 2, SEQ ID NO: 3 and SEQ ID NO: 4; and/or the amino acid sequences of the LCDR1, LCDR2 and LCDR3 respectively include SEQ ID NO: 6, SEQ ID NO: 7 and SEQ ID NO: 8. In specific examples, the amino acid sequence of the VH domain is at least 90% identical to SEQ ID NO: 1 and includes the HCDR1, HCDR2 and HCDR3 sequence of SEQ ID NO: 1; and/or the amino acid sequence of the VL domain is at least 90% identical to SEQ ID NO: 5 and includes the LCDR1, LCDR2 and LCDR3 sequences of SEQ ID NO: 5. In one non-limiting example, the amino acid sequence of the VH domain comprises or consists of SEQ ID NO: 1; and/or the amino acid sequence of the VL domain comprises or consists of SEQ ID NO: 5.

In some examples, the monoclonal antibody is a mammalian immunoglobulin, such as an IgG, IgM, IgE, IgD or IgA. In other examples, the monoclonal antibody is an antigen-binding fragment, such as but not limited to a Fab fragment, a Fab′ fragment, a F(ab)′₂ fragment, a single chain variable fragment (scFv) or a disulfide stabilized variable fragment (dsFv). Immunoglobulin molecules and antigen-binding fragments are further discussed in section V.

In some examples, the anti-FXI antibody is administered at a dose of about 0.1 mg/kg, about 0.5 mg/kg, about 1.0 mg/kg, about 2.5 mg/kg or about 5.0 mg/kg. The antibody can be administered in a single dose or multiple doses, such as daily, every other day, bi-weekly or weekly.

In particular aspects, the agent that inhibits activation of FXI or inhibits activity of FXIa is a small molecule inhibitor of FXI or FXIa. In some examples, the small molecule inhibitor is asundexian or milvexian.

In other particular aspects, the agent that inhibits expression of FXI is an antisense compound that specifically hybridizes with a nucleic acid sequence encoding FXI, such as an FXI mRNA. FXI nucleic acid sequences are well-known and available in public databases (see, e.g., NCBI Gene ID 2160). In some examples, the antisense compound is an antisense oligonucleotide (ASO), a small inhibitory RNA (siRNA), or a microRNA (miRNA).

In some aspects of the disclosed methods, the agent is administered in a composition that includes a pharmaceutically acceptable carrier. In some examples, the composition is formulated for intravenous administration. In some examples, the composition is administered daily, every other day, bi-weekly or weekly. Compositions, such as pharmaceutical compositions that include an agent that inhibits activation of FXI, inhibits the activity of activated FXI (FXIa), or reduces expression of FXI (such as a monoclonal antibody, small molecule or antisense compound) can be formulated with an appropriate solid or liquid carrier, depending upon the particular mode of administration chosen. The pharmaceutically acceptable carriers and excipients useful in this disclosure are conventional (scc, e.g., Remington: The Science and Practice of Pharmacy, 22^{nd} ed., London, UK: Pharmaceutical Press, 2013). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate.

In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations. The dosage form of the pharmaceutical composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (for example, solutions or suspensions) and include mists, sprays and the like. Oral formulations can be liquid (for example, syrups, solutions or suspensions), or solid (for example, powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known.

In some aspects of the disclosed methods, the neuroinflammation being treated includes encephalopathy or demyelination.

In some aspects, the subject being treated has an autoimmune disease of the central nervous system (CNS). In some examples, the autoimmune disease is multiple sclerosis (MS), neuromyelitis optica (NMO), anti-myelin oligodendrocyte glycoprotein antibody disease (MOG), autoimmune encephalitis, acute disseminated encephalomyelitis (ADEM), chronic meningitis, central nervous system vasculitis, or Hashimoto's encephalitis. In particular examples, the autoimmune disease is MS. In specific non-limiting examples, the MS is relapsing-remitting multiple sclerosis (RRMS), secondary-progressive multiple sclerosis (SPMS), or primary progressive multiple sclerosis (PPMS).

In some aspects, the subject being treated has a neurodegenerative disease. In some examples, the neurodegenerative disease is amyotrophic lateral sclerosis (ALS), Parkinson's disease, Alzheimer's disease, Huntington's disease, Lewy body dementia, vascular dementia, progressive supranuclear palsy, corticobasal degeneration, multiple system atrophy, frontotemporal dementia, or ischemic stroke.

### V. Monoclonal Antibodies and Antigen-Binding Fragments Thereof

In some aspects of the disclosed methods, the monoclonal antibody is a mammalian immunoglobulin, such as an IgG, IgM, IgE, IgD or IgA. The antibodies can further be any subtype. For example, IgG antibodies include subtypes IgG₁, IgG₂ (including IgG₂ₐ and IgG_{2b}), IgG₃ and IgG₄. The class of an antibody that specifically binds FXI can be switched with another. In one aspect, a nucleic acid molecule encoding V_{H} or V_{L} (e.g., SEQ ID NO: 9 or SEQ ID NO: 10) is synthesized or isolated using methods well-known in the art, such that it does not include any nucleic acid sequences encoding the constant region of the light or heavy chain, respectively. The nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds FXI that was originally IgG may be class switched to an IgM. Class switching can also be used to convert one IgG subclass to another, such as from IgG₁ to IgG₂.

In other aspects, the monoclonal antibody is an antigen-binding fragment, such as but not limited to a Fab fragment, a Fab′ fragment, a F(ab)′₂ fragment, a single chain variable fragment (scFv) or a disulfide stabilized variable fragment (dsFv). These antibody fragments retain the ability to selectively bind with the antigen. These fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the variable region of the light chain and the variable region of the heavy chain expressed as two chains;
(5) Single chain antibody (such as scFv), defined as a genetically engineered molecule containing the variable region of the light chain, the variable region of the heavy chain, linked by a suitable polypeptide linker as a genetically fused single chain molecule; and
(6) A dimer of a single chain antibody (scFV₂), defined as a dimer of a scFv. This has also been termed a “miniantibody.”

Methods of making these fragments are known (see for example, Harlow and Lane, Antibodies: A Laboratory Manual, Cold Spring Harbor Laboratory, New York, 1988).

In additional aspects, the antibodies are Fv antibodies, which are typically about 25 kDa and contain a complete antigen-binding site with three CDRs in each heavy chain and each light chain. To produce these antibodies, the V_{H} and the V_{L} can be expressed from two individual nucleic acid constructs (e.g., SEQ ID NO: 9 and SEQ ID NO: 10) in a host cell. If the V_{H} and the VI, are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} domains connected by a peptide linker. These single-chain antigen binding proteins (scFv) are prepared by constructing a structural gene comprising DNA sequences encoding the V_{H} and V_{L} domains (e.g., SEQ ID NO: 9 and SEQ ID NO: 10) connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993). Dimers of a single chain antibody (scFV₂) are also contemplated.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will understand that conservative variants of the antibodies can be produced. Such conservative variants employed in antibody fragments, such as dsFv fragments or in scFv fragments, will retain the amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions, for example conservative substitutions) can be made in the V_{H} and the V_{L} regions to increase yield.

### EXAMPLES

The following examples are provided to illustrate particular features of certain aspects of the disclosure, but the scope of the claims should not be limited to those features exemplified.

### Example 1: Materials and Methods

This example describes the materials and experimental procedures for Examples 2-5.

### Reagents

The anti-FXI monoclonal antibody, 14E11, was generated and purified as previously described in PCT Publication No. WO 2010/080623 (see also Cheng et al., Blood 116 (19): 3981-3989, 2010). The amino acid sequences of the 14E11 VH and VL domains, along with the VH and VL domain CDR sequences, are provided below. Exemplary nucleic acid sequences encoding the VH and VL domains are also listed below.

14E11 VH domain amino acid sequence (SEQ ID NO: 1)
QVQLEESGPGLVAPSQSLSITCTVSGFSLTGYGIYWVRQPPGKGLEWLGMIWGDGRTDYNS
ALKSRLSISKDNSKSQVFLKMNSLQTDDTARYYCARDYYGSKDYWGQGTTLTVSS
14E11 VL domain amino acid sequence (SEQ ID NO: 5)
DIVMTQSHKFMSTSVGDRVSITCKASQDVSTAVAWYQQKPGQSPKLLIYLTSYRNTGVPD
RFTGSGSGTDFTFTISSVQAEDLAVYYCQQHYKTPYSFGGGTKLERLR

| CDR | Sequence | SEQ ID NO: |
|---|---|---|
| H-CDR1 | GYGIY | 2 |
| H-CDR2 | MIWGDGRTDYNSALKSRLS | 3 |
| H-CDR3 | DYYGSKDY | 4 |
| L-CDR1 | KASQDVSTAVA | 6 |
| L-CDR2 | LTSYRNTGVPDRFT | 7 |
| L-CDR3 | HYKTPYSF | 8 |

14E11 VH domain nucleic acid sequence (SEQ ID NO: 9)
CAGGTGCAGCTGGAGGAGTCAGGACCTGGCCTGGTGGCGCCCTCACAGAGCCTGTCCA
TCACATGCACCGTCTCAGGGTTCTCATTAACCGGCTATGGTATATACTGGGTTCGCCAG
CCTCCAGGAAAGGGTCTGGAGTGGCTGGGAATGATATGGGGTGATGGAAGAACAGAC
TATAATTCAGCTCTCAAATCCAGACTGAGCATCAGTAAGGACAACTCCAAGAGCCAAG
TTTTCTTAAAAATGAACAGTCTGCAAACTGATGACACAGCCAGGTACTACTGTGCCAG
AGATTACTACGGTAGTAAGGACTACTGGGGCCAAGGCACCACTCTCACAGTCTCCTCA
14E11 VL domain nucleic acid sequence (SEQ ID NO: 10)
GACATTGTGATGACCCAGTCTCACAAATTCATGTCCACATCAGTAGGAGACAGGGTCA
GCATCACCTGCAAGGCCAGTCAGGATGTGAGTACTGCTGTTGCCTGGTATCAACAGAA
ACCAGGACAATCTCCTAAACTACTGATTTACTTGACATCCTACCGGAACACTGGAGTCC
CTGATCGCTTCACTGGCAGTGGATCTGGGACGGATTTCACTTTCACCATCAGCAGTGTG
CAGGCTGAAGACCTGGCAGTTTACTACTGTCAGCAACATTATAAAACTCCGTATTCGTT
CGGAGGGGGGACCAAGCTGGAACGGTTACGG

### Animals

The male C57BL/6 mice used in this study were housed at the Portland Veterans Affairs Medical Center in the Animal Resource Facility (Portland, OR, US) according to guidelines set by the National Research Council and the Committee on Care and Use of Laboratory Animals of the Institute of Laboratory Animal Resources.

### EAE Induction and 14E11 Treatment

EAE was induced by inoculating C57BL/6 mice subcutaneously with mouse myelin oligodendrocyte glycoprotein, peptides 35-55 (MOG35-55) combined with complete Freund's adjuvant containing heat-inactivated M. tuberculosis, as previously described (Sinha et al., J Neuroimmune Pharmacol 5 (2): 231-239, 2010). On days 0 and 2 relative to immunization, mice were intraperitoneally injected with 75 ng and 200 ng of pertussis toxin (Ptx), respectively. To quantify the symptoms of EAE, a disease score was assigned to each mouse daily. The scale used to assess EAE symptoms was as follows: 0, normal; 1, limp tail or mild hind limb weakness; 2, moderate hind limb weakness or mild ataxia; 3, moderately severe hind limb weakness; 4, severe hind limb weakness or mild forelimb weakness or moderate ataxia; 5, paraplegia with no more than moderate forelimb weakness; and 6, paraplegia with severe forelimb weakness or severe ataxia or moribund condition.

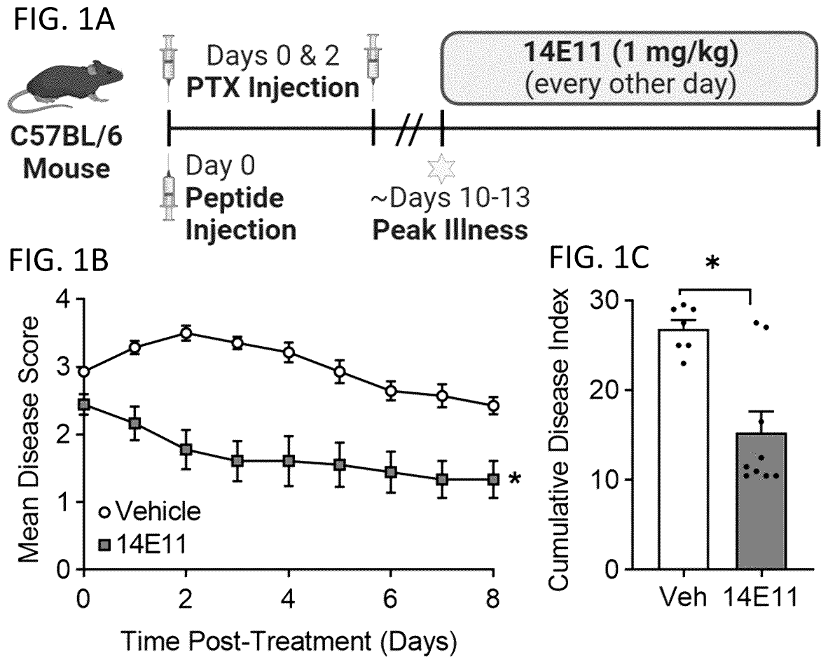

At the onset of clinical signs of EAE (disease score ≥2.5, typically between days 10-13), mice were randomized into two groups. The mice were then administered either the anti-FXI antibody, 14E11 (1 mg/kg, n=9), or vehicle (saline, n=7) at the same volume intravenously (FIG. 1A). The 14E11 dose level was selected based on previous work showing that administration of 14E11 at similar doses produced a sustained anticoagulant effect, as evidenced by prolonged clotting times (Cheng et al., Blood 116 (19): 3981-3989, 2010; Leung et al., Transl Stroke Res 3 (3): 381-389, 2012; Ngo et al., J Thromb Haemost 19 (4): 1001-1017, 2021; Tucker et al., Blood 119 (20): 4762-4768, 2012). The mice were monitored for changes in disease score by an investigator blinded to treatment status until mice were euthanized for ex vivo analyses.

### Flow Cytometry

Flow cytometry was used to measure markers of inflammation in the CNS and periphery after euthanasia. To quantify the population of mononuclear cells in the CNS, Percoll density gradient centrifugation was used to isolate mononuclear cells (Bebo et al., J Neurosci Res 45 (6): 680-689, 1996). The CNS cells (n=3 per group) were then pooled to achieve sufficient numbers for the antibody staining protocol. To measure the activation of the macrophage subpopulation of splenocytes, spleen tissue was homogenized and single cell suspensions were prepared. Cells (1×10⁶) were washed with a staining media comprised of PBS with NaN₃ (0.1%) and BSA (1%) and stained with combinations of anti-CD4, anti-CD45, anti-ICAM-1 and anti-CD11b antibodies. These antibodies were obtained from sources previously described (Dziennis et al., Brain Behav Immun 25 (4): 685-695, 2011).

### Immunohistochemistry

To perform ex vivo analyses following vehicle or 14E11 treatment, mice were euthanized with an isoflurane overdose until respiration ceased. They were then heparinized, transcardially perfused with 100 mL of 4% paraformaldehyde (mass/volume in sodium phosphate buffer [0.1 M, pH 7.4]), and fixed at 4° C. for 24 hours. Mice then underwent necropsy and intact spinal columns were removed. Spinal cord sections 1-2 mm in length were dissected from the thoracic and lumbar regions from one representative animal per treatment group to analyze CNS pathology and fibrin(ogen) accumulation, respectively.

To assess CNS pathology, thoracic spinal cord tissues were re-fixed in 5% glutaraldehyde (mass/volume in sodium phosphate buffer [0.1 M, pH 7.4]) at 4° C. for 72 hours, post-fixed in 1% osmium tetroxide for 3.5 hours, rehydrated in ethanol, and embedded in plastic. Then, 0.5 μm semithin sections were obtained with a microtome, mounted onto pre-cleaned microscope slides, and stained with toluidine blue to visualize nerve structures. Spinal sections were imaged at 20× and manually stitched into a complete composite of the section. To assess for axonal damage within the thoracic tissue, composite images were analyzed by a trained, blinded user as described previously (Wang et al., J Neurochem 98 (6): 1817-1827, 2006). Briefly, regions of interest within the white matter tracts were segmented for areas of tissue damage, which included demyelinated axons, degenerating axons, and disrupted compact myelin. Percent area of tissue damage was quantified by measuring area of damaged white matter and total area of white matter (damaged and intact).

To assess fibrin(ogen) accumulation within the CNS, lumbar spinal cord tissues were fixed and embedded in paraffin for sectioning. Sections were blocked in 10% normal goat serum containing 1% bovine serum albumin and 0.025% Triton-X at room temperature for 45 minutes to decrease nonspecific staining. Tissue sections were then incubated with a primary antibody to fibrin(ogen) (1:50 in goat serum, rabbit polyclonal, MP Biomedicals) overnight at 4° C. followed by goat anti-rabbit IgG ALEXA FLUOR 488 (Molecular Probes). Slides were rinsed, mounted in aqueous media, and imaged at 20× with a Zeiss Axiovert fluorescent microscope. 20× images were processed identically in SlideBook (ver. 5.5) and manually stitched into a composite of the entire section using PHOTOSHOP. Quantitative analysis of fibrin(ogen) accumulation was performed on composite images using ImageJ (ver. 2.3.0). Color channels were split, and the green channel intensity histogram data were obtained and utilized to determine the background threshold value. Mean gray values corresponding to fibrin(ogen) signal were measured above the threshold value. Corrected integrated density was determined by subtracting background and accounting for area of fibrin(ogen).

### Statistical Analysis

Shapiro-Wilk and F-tests were used to test for normality and compare variances in the data. If the data were normally distributed with equal standard deviations, unpaired t-tests were used to compare vehicle and 14E11-treated mice. If the data were not normally distributed, the data were compared by a Mann-Whitney test. A P value less than 0.05 was considered statistically significant. GraphPad Prism 9 (ver. 9.5.0) was used to perform statistical analyses.

### Example 2: Effect of FXI Inhibition on Clinical Signs of EAE

Patients with MS experience discrete episodes, also referred to as ‘attacks’ or ‘relapses’, during which they present with varying clinical symptoms (e.g., changes in gait, muscle weakness, and incoordination) (Gelfand, Handb Clin Neurol 122:269-290, 2014). Clinical rating scales are commonly used to assess the severity of neurological dysfunction and inform treatment strategies in humans, as well as monitor disease progression in animal models (Hulleck et al., Front Med Technol 4:901331, 2022; Noseworthy, Ann Neurol 36 Suppl: S80-85, 1994). To quantify the effects of FXI inhibition on EAE symptoms, EAE mice were administered 14E11 or vehicle control at peak disease every other day for four days (FIG. 1A). Mice treated with 14E11 had an average peak disease score of 2.4, whereas mice in the control cohort had an average peak disease score of 3.5, which represents a 31.4% reduction (FIG. 1B). To further quantify this effect, cumulative disease index (CDI) was calculated by summing daily disease scores for each cohort. Mice treated with 14E11 had a mean CDI of 15.3, while mice in the control group had a mean CDI of 26.9, representing a 43% decrease in mean CDI with 14E11 treatment (FIG. 1C). These data confirm that targeting FXI reduced clinical symptom severity in a mouse model of MS. No bleeding in mice was observed during this study, nor during hemostatic evaluations in previous mouse studies using 14E11 (Leung et al., Transl Stroke Res 3 (3): 381-389, 2012; Tucker et al., Blood 119 (20): 4762-4768, 2012).

### Example 3: Effect of FXI Inhibition on Clinical Signs of EAE

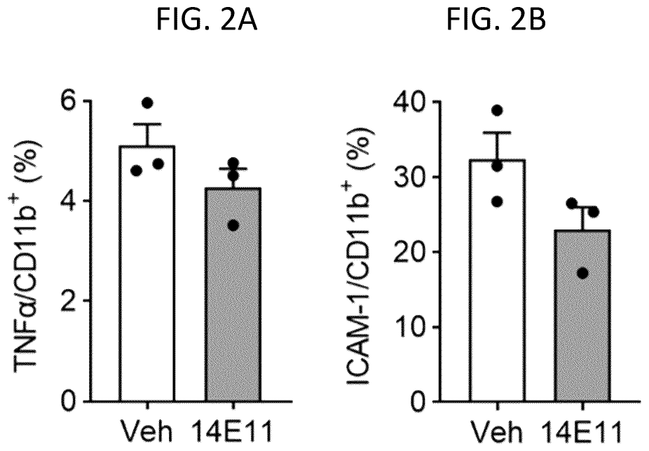

Since activated macrophages and microglia have been shown to upregulate demyelination in the early stages of MS (Wang et al., Front Pharmacol 10:286, 2019), the role of FXI was next evaluated in mediating the inflammatory response in EAE. To do this, macrophage subpopulation activation was measured in splenocytes harvested from mice with EAE. No differences were observed for CD11b⁺ macrophages expressing TNFα or ICAM-1 following treatment with 14E11 (FIGS. 2A & 2B).

The population of activated macrophages/microglia (CD11b/CD45^{high} cells) and CD4+ T-cells in brain samples from vehicle control and 14E11 treated mice was then quantified. To overcome the lower limit of CNS cells required for the antibody staining protocol, the CNS cells (n=3 per group) were pooled (n=1 per group) before flow cytometry analyses. Compared to the vehicle-treated group, total numbers of mononuclear cells recovered from the 14E11 treated group were reduced by ˜50% (from 3.2M to 1.6M cells) and absolute numbers of infiltrating CD11b/CD45^{high} cells in the CNS were reduced by 24% (from 188,800 to 142,880 cells) and CD4⁺ T cells by 44% (from 115,520 to 65,120 cells), respectively. These results indicate that FXI plays a substantial role in reducing EAE-induced inflammation in the CNS.

### Example 4: Effect of FXI Inhibition on Demyelination in the Thoracic Spinal Cord

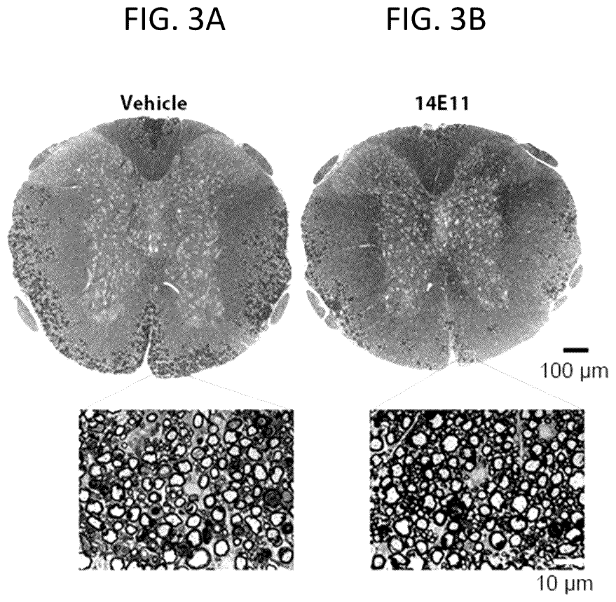

The progression of MS is marked by increasing instances of demyelinated lesions in the CNS that result in the debilitating loss of nervous system function (Gelfand, Handb Clin Neurol 122:269-290, 2014). To determine the effect of FXI inhibition on myelination, spinal cord cross sections were stained with toluidine blue and light microscopy was used to assess myelination. As shown in FIGS. 3A-3B, spinal cord sections from mice treated with 14E11 demonstrated a marked reduction in demyelination compared to mice in the control cohort. The demyelination damage was quantified as percent of damaged white matter in a representative spinal cord image (n=1) from each group. The percent of white matter area damaged was reduced from 13.6% in vehicle treated controls to 2.14% in 14E11 treated mice, representing an 84.3% decrease in damage with 14E11 treatment. These results demonstrate that targeting FXI preserved myelination in the spinal cords of EAE mice.

### Example 5: Fibrin (Ogen) Accumulation in the CNS

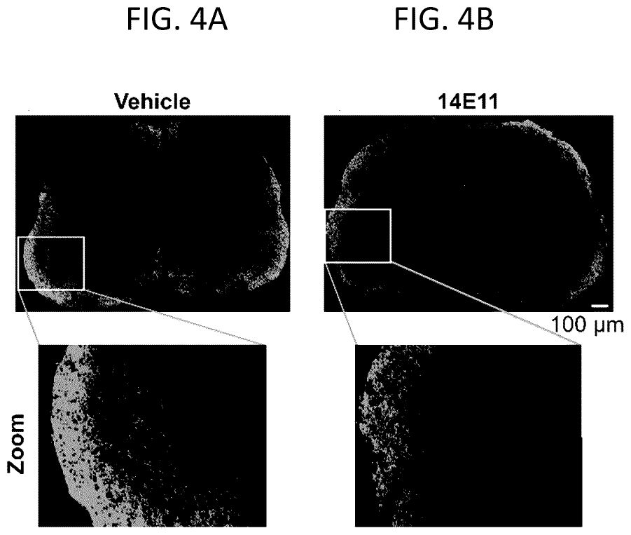

Accumulation of the plasma protein fibrin(ogen) in the CNS is a prominent feature of BBB disruption and MS pathology. Studies have shown that fibrinogen accumulation correlates with axonal damage in the mouse models of EAE (Davalos et al., Nat Commun 3 (1): 1227, 2012). Since there was a reduction in the percent area of damaged white matter in this study, the effects of FXI inhibition on fibrin(ogen) deposition in the lumbar region of the CNS was next investigated (FIGS. 4A-4B). Fibrin (ogen) accumulation was quantified as the percent area positive in representative images of the lumbar region (n=1 per group). The fibrin(ogen) signal was reduced from 4.88% in the vehicle control to 2.55% for 14E11 treatment. Additionally, the corrected fluorescent integrated density reduced from 5.48×10⁷ RFUs to 2.59×10⁷ RFUs for vehicle and 14E11 treatments, respectively. This corresponds to a 52.7% reduction in intensity of fibrin(ogen) signal for 14E11 treatment compared to vehicle control.

It will be apparent that the precise details of the methods or compositions described may be varied or modified without departing from the spirit of the described aspects of the disclosure. We claim all such modifications and variations that fall within the scope and spirit of the claims below.

1. A method of treating neuroinflammation in a subject, comprising administering to the subject a therapeutically effective amount of an agent that inhibits activation of coagulation factor XI (FXI), inhibits the activity of activated FXI (FXIa), or reduces expression of FXI, thereby treating neuroinflammation in the subject.

2. The method of claim 1, wherein the agent that inhibits activation of FXI, inhibits activity of FXIa, or reduces expression of FXI is a monoclonal antibody, a small molecule inhibitor, or an antisense compound.

3. The method of claim 1, wherein:
the agent that inhibits activation of FXI is a monoclonal antibody specific for FXI;
the agent that inhibits activation of FXI or inhibits activity of FXIa is a small molecule inhibitor of FXI or FXIa; or
the agent that inhibits expression of FXI is an antisense compound that specifically hybridizes with a nucleic acid sequence encoding FXI.

4. The method of claim 3, wherein the monoclonal antibody specific for FXI binds the A2 domain of FXI.

5. The method of claim 3, wherein the monoclonal antibody specific for FXI comprises a variable heavy (VH) domain and a variable light (VL) domain, wherein:
the VH domain comprises the heavy chain complementarity determining region 1 (HCDR1), HCDR2 and HCDR3 sequences of SEQ ID NO: 1; and
the VL domain comprises the light chain complementarity determining region 1 (LCDR1), LCDR2, and LCDR3 sequence of SEQ ID NO: 5.

6. The method of claim 5, wherein:
the amino acid sequences of the HCDR1, HCDR2 and HCDR3 respectively comprise SEQ ID NO: 2, SEQ ID NO: 3 and SEQ ID NO: 4; and
the amino acid sequences of the LCDR1, LCDR2 and LCDR3 respectively comprise SEQ ID NO: 6, SEQ ID NO: 7 and SEQ ID NO: 8.

7. The method of claim 5, wherein:
the amino acid sequence of the VH domain is at least 90% identical to SEQ ID NO: 1 and comprises the HCDR1, HCDR2 and HCDR3 sequence of SEQ ID NO: 1; and
the amino acid sequence of the VL domain is at least 90% identical to SEQ ID NO: 5 and comprises the LCDR1, LCDR2 and LCDR3 sequences of SEQ ID NO: 5.

8. The method of claim 5, wherein:
the amino acid sequence of the VH domain comprises or consists of SEQ ID NO: 1; and
the amino acid sequence of the VL domain comprises or consists of SEQ ID NO: 5.

9. The method of claim 3, wherein:
the monoclonal antibody is an IgG; or
the monoclonal antibody is a Fab fragment, a Fab′ fragment, a F(ab)′₂ fragment, a single chain variable fragment (scFv) or a disulfide stabilized variable fragment (dsFv).

10. The method of claim 3, wherein the small molecule inhibitor is asundexian or milvexian.

11. The method of claim 3, wherein the antisense compound is an antisense oligonucleotide (ASO), a small inhibitory RNA (siRNA), or a microRNA (miRNA).

12. The method of claim 1, wherein the agent is administered in a composition comprising a pharmaceutically acceptable carrier.

13. The method of claim 12, wherein the composition is formulated for intravenous administration.

14. The method of claim 1, wherein the neuroinflammation comprises encephalopathy or demyelination.

15. The method of claim 1, wherein the subject has an autoimmune disease of the central nervous system (CNS).

16. The method of claim 15, wherein the autoimmune disease of the CNS is multiple sclerosis (MS), neuromyelitis optica (NMO), anti-myelin oligodendrocyte glycoprotein antibody disease (MOG), autoimmune encephalitis, acute disseminated encephalomyelitis (ADEM), chronic meningitis, central nervous system vasculitis, or Hashimoto's encephalitis.

17. The method of claim 16, wherein the autoimmune disease of the CNS is MS.

18. The method of claim 17, wherein the MS is relapsing-remitting multiple sclerosis (RRMS), secondary-progressive multiple sclerosis (SPMS), or primary progressive multiple sclerosis (PPMS).

19. The method of claim 1, wherein the subject has a neurodegenerative disease.

20. The method of claim 19, wherein the neurodegenerative disease is amyotrophic lateral sclerosis (ALS), Parkinson's disease, Alzheimer's disease, Huntington's disease, Lewy body dementia, vascular dementia, progressive supranuclear palsy, corticobasal degeneration, multiple system atrophy, frontotemporal dementia, or ischemic stroke.


SEQUENCE LISTING (10 sequences)

SEQ ID NO: 1 - protein, 116 aa, synthetic construct
QVQLEESGPGLVAPSQSLSITCTVSGFSLTGYGIYWVRQPPGKGLEWLGMIWGDGRTDYNSALKSRLSISKDNSKSQVFLKMNSLQTDDTARYYCARDYYGSKDYWGQGTTLTVSS

SEQ ID NO: 2 - protein, 108 aa, synthetic construct
DIVMTQSHKFMSTSVGDRVSITCKASQDVSTAVAWYQQKPGQSPKLLIYLTSYRNTGVPDRFTGSGSGTDFTFTISSVQAEDLAVYYCQQHYKTPYSFGGGTKLERLR

SEQ ID NO: 3 - protein, 5 aa, synthetic construct
GYGIY

SEQ ID NO: 4 - protein, 19 aa, synthetic construct
MIWGDGRTDYNSALKSRLS

SEQ ID NO: 5 - protein, 8 aa, synthetic construct
DYYGSKDY

SEQ ID NO: 6 - protein, 11 aa, synthetic construct
KASQDVSTAVA

SEQ ID NO: 7 - protein, 14 aa, synthetic construct
LTSYRNTGVPDRFT

SEQ ID NO: 8 - protein, 8 aa, synthetic construct
HYKTPYSF

SEQ ID NO: 9 - DNA, 348 bp, synthetic construct
  source 1..348: mol_type = other DNA
caggtgcagctggaggagtcaggacctggcctggtggcgccctcacagagcctgtccatcacatgcaccgtctcagggttctcattaaccggctatggtatatactgggttcgccagcctccaggaaagggtctggagtggctgggaatgatatggggtgatggaagaacagactataattcagctctcaaatccagactgagcatcagtaaggacaactccaagagccaagttttcttaaaaatgaacagtctgcaaactgatgacacagccaggtactactgtgccagagattactacggtagtaaggactactggggccaaggcaccactctcacagtctcctca

SEQ ID NO: 10 - DNA, 324 bp, synthetic construct
  source 1..324: mol_type = other DNA
gacattgtgatgacccagtctcacaaattcatgtccacatcagtaggagacagggtcagcatcacctgcaaggccagtcaggatgtgagtactgctgttgcctggtatcaacagaaaccaggacaatctcctaaactactgatttacttgacatcctaccggaacactggagtccctgatcgcttcactggcagtggatctgggacggatttcactttcaccatcagcagtgtgcaggctgaagacctggcagtttactactgtcagcaacattataaaactccgtattcgttcggaggggggaccaagctggaacggttacgg